(19) United States
(12) Patent Application Publication
(10) Pub. No.: US 2022/0340687 A1
(43) Pub. Date: Oct. 27, 2022

(54) METHODS OF TREATMENT USING OMALIZUMAB

(71) Applicants: NOVARTIS AG, Basel (CH); GENENTECH, INC., South San Francisco, CA (US)

(72) Inventors: XAVIER JAUMONT, Madeleine (CH); Oscar PALOMARES, Madrid (ES)

(21) Appl. No.: 17/642,319

(22) PCT Filed: Aug. 31, 2020
(86) PCT No.: PCT/IB2020/058097

§ 371 Date: Mar. 11, 2022

(30) Foreign Application Priority Data
Sep. 13, 2019 (EP) 19197285.0

(51) Int. Cl. C07K 16/42; A61K 45/06; A61K 39/395; A61P 37/08

(52) U.S. Cl. CPC C07K 16/4291; A61K 45/06; A61K 39/39566; A61P 37/08; C07K 2317/24

(57) ABSTRACT

The present disclosure relates to methods for modifying the course of a disease or disorder involving Treg cells dysfunctioning, in particular in patients having an allergic disease or condition.

### TECHNICAL FIELD

The present disclosure relates to methods of preventing or treating diseases or disorders involving dyfunctioning of regulatory T cells, using an anti-IgE antibody, e.g., omalizumab.

### BACKGROUND OF THE DISCLOSURE

T cells are lymphocytes that develop in the thymus gland and play a central role in the immune response. These cells can be distinguished from other lymphocytes by the presence of a T-cell receptor on their surface. They originate as precursor cells derived from bone marrow, and develop into several distinct types of T cells once they have migrated into the thymus.

Regulatory T cells (Treg cells), also called suppressor T cells, are a subpopulation of T cells that, when induced, modulate the immune system to prevent pathological self-reactivity and maintain tolerance to self-antigens (whereby the immune system is able to distinguish invading cells from self-cells) and by thus are involved in preventing autoimmune diseases. Induced Treg cells, are immunomodulators: they actively suppress inappropriate immune responses by suppressing (or downregulating) induction and proliferation of the effector T cells.

Functional regulatory T cells can be induced by dendritic cells (DCs).

Immunoglobulin E (IgE) is an antibody associated with hypersensitivity and allergic reactions. IgE mainly binds on the high-affinity IgE receptor (FIERI) on mast cells, basophils and dendritic cells and hence decreases the induction of the regulatory T cells. This may create conditions for diseases which depend on the induction of the Treg cells, to be triggered or exacerbated. Different diseases may be listed such as asthma, myasthenia gravis, rhumatoid arthritis, lupus, inflammatory bowel diseases like Crohn disease, different inflamatory endocrinopathies.

Despite existing medications, these diseases are still not fully controlled and their treatments (systemic corticosteroids, immumosuppressors) have a known low safety profile. There is a need of identifying better treatments for these diseases, or at least to find alternative therapies that would permit administering lower doses of corticosteroids or immumosuppressors.

Xolair® (omalizumab) is a recombinant DNA-derived humanized monoclonal antibody that selectively binds to free, circulating human immunoglobulin E (IgE) thus inhibiting IgE binding to IgE receptors on the surface of mast cells and basophils resulting in decreased release of allergic mediators. By binding to free, circulating IgE, omalizumab also lowers serum free IgE levels and down-regulates the number of IgE receptors on the surface of mast cells and basophils. Omalizumab widely used for the treatment of Allergic asthma, allergic rhinitis and chronic spontaneous urticaria (CSU).

### SUMMARY OF THE DISCLOSURE

We have now discovered that omalizumab is able to promote the induction of regulatory T cells. The identification of this new mode of action of omalizumab opens the route of new treatment possibilities for many diseases or disorders involving dyfunctioning of regulatory T cells, in particular autoimmune diseases or disorders and cancer.

Accordingly, disclosed herein are methods of preventing or treating diseases or disorders involving dyfunctioning of regulatory T cells, comprising administering a therapeutically effective amount of an anti-IgE antibody, e.g an anti-IgE antibody that selectively binds to free, circulating human IgE (e.g., omalizumab), to a patient in need thereof.

### BRIEF DESCRIPTION OF THE FIGURES

FIG. 1. Characterization of purified pDCs from human PBMC. 0.2×106-106 pDCs (85-95% purity) were isolated from 200×106 PBMCs.

A) Representative dot-plots showing the purity before and after the pDCs isolation. Purified pDCs were HLADR+CD303+CD304+CD123+.
B) Non-linear correlation between the frequency of pDCs with FcεR1-bound IgE (% IgE+pDCs) and plasma IgE, “r” Spearman correlation coefficient (n=24).
C) Plasma IgE levels contained in IgE-pDCs and IgE+pDCs donors (n=24). Those donors whose pDCs were >45% positive for FcεR1-bound IgE, were considered IgE+pDCs donors. Values are given as mean±SEM. **p<0.01 in paired t-test comparing IgE-pDCs and IgE+pDCs donors.

FIG. 2. IgE-mediated FcεR1-crosslinking alters the expression of genes, markers and cytokine secretion in IgE+pDCs. pDCs were purified from atopic donors and cultured in RPMI medium plus 10 ng/mL IL-3, 2 μM TLR9-L and 10 μg/mL IgE-FcεR1-crosslinker (CL) or isotype control (IgG) for 18 hours.

A) From left to right, frequency of CD83 (n=4), CD86 (n=4), HLA-DR mean fluorescence intensity (MFI, n=4), frequency of PD-L1 (n=8) and OX40L (n=8) gated in pDCs.
B) Expression of ICOSL, PD-L1 and IDO genes (n=8).
C) IFN-α (n=5) and TNF-α (n=6) measured in culture supernatant.

Values are given as mean±SEM. ***p<0.001, **p<0.01, *p<0.05 in paired t-test comparing multiple conditions.

FIG. 3. IgE-FcεR1-crosslinker impairs pDCs capacity to induce Treg cells. pDCs were purified from atopic donors and cultured in RPMI medium plus 10 ng/mL IL-3 and 2 μM TLR9-L for 18 hours. Afterwards, pDCs were washed and cocultured with naïve CD4+ T cells (1:5 ratio) in RPMI medium plus 10 ng/mL IL-3, 10 μg/mL IgE-FcεR1-crosslinker (CL) or isotype control (IgG) for 5 days.

A) IFN-α, IL-5, IL-2 and IL-10 cytokines in coculture supernatant (n=9).
B) graph of CD127lowCD25+Foxp3+ Treg cells frequency gated over CD4+ cells (n=9). Values are mean±SEM. ***p<0.001, **p<0.01, *p<0.05 paired t-test in multiple conditions.

FIG. 4. Omalizumab (Oma) removes membrane-bound IgE from purified pDCs. pDCs from atopic donors were purified and cultured with IL-3 and 5, 10 and mg/mL of Oma for 24 hours.

A) Left: Frequency of FcεR1α-bound IgE+ cells gated in pDCs. Right: Frequency of cells with unoccupied FcεR1, gated in pDCs (n=4).
B) Viability of pDCs after 24 hours incubation with Oma (n=2). Values are given as mean±SEM. **p<0.01, *p<0.05 in paired t-test comparing multiple conditions.

### DETAILED DESCRIPTION OF THE DISCLOSURE

### Definitions

As used herein, IgE refers to Immunoglobulin E.

The term “comprising” encompasses “including” as well as “consisting,” e.g., a composition “comprising” X may consist exclusively of X or may include something additional, e.g., X+Y.

The term “about” in relation to a numerical value x means, for example, +/−10%. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The word “substantially” does not exclude “completely,” e.g., a composition which is “substantially free” from Y may be completely free from Y. Where necessary, the word “substantially” may be omitted from the definition of the disclosure.

The term “antibody” as referred to herein includes naturally-occurring and whole antibodies. A naturally-occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed hypervariable regions or complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” of an antibody, as used herein, refers to fragments of an antibody that retain the ability to specifically bind to IgE. It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, CL and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated CDR. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies are also intended to be encompassed within the term “antibody”. Single chain antibodies and antigen-binding portions are obtained using conventional techniques known to those of skill in the art.

The term “K_{D}” is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, or using a biosensor system such as a Biacore® system. In some embodiments, the anti-IgE antibody or antigen-binding fragment thereof according to the invention, e.g., omalizumab, binds human IgE with a K_{D} of about 0.02 to 7.7 nM, e.g. 100-250 pM.

The term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity. Standard assays to evaluate the binding affinity of the antibodies toward IgE of various species are known in the art, including for example, ELISAs, western blots and RIAs. The binding kinetics (e.g., binding affinity) of the antibodies also can be assessed by standard assays known in the art, such as by Biacore analysis.

The term “derivative”, unless otherwise indicated, is used to define amino acid sequence variants, and covalent modifications (e.g. pegylation, deamidation, hydroxylation, phosphorylation, methylation, etc.) of an anti-IgE antibody or antigen-binding fragment thereof, e.g., omalizumab, according to the present disclosure, e.g., of a specified sequence (e.g., a variable domain). A “functional derivative” includes a molecule having a qualitative biological activity in common with the disclosed anti-IgE antibodies. A functional derivative includes fragments and peptide analogs of an anti-IgE antibody as disclosed herein. Fragments comprise regions within the sequence of a polypeptide according to the present disclosure, e.g., of a specified sequence.

The phrase “substantially identical” means that the relevant amino acid or nucleotide sequence (e.g., V_{H} or V_{L} domain) will be identical to or have insubstantial differences (e.g., through conserved amino acid substitutions) in comparison to a particular reference sequence. Insubstantial differences include minor amino acid changes, such as 1 or 2 substitutions in a 5 amino acid sequence of a specified region (e.g., V_{H} or V_{L} domain). In the case of antibodies, the second antibody has the same specificity and has at least 50% of the affinity of the same. Sequences substantially identical (e.g., at least about 85% sequence identity) to the sequences disclosed herein are also part of this application. In some embodiments, the sequence identity of a derivative anti-IgE antibody (e.g., a derivative of omalizumab, e.g., an omalizumab biosimilar antibody) can be about 90% or greater, e.g., 90%, 91%, 92%, 93%, 94%, 95%, 96%, 97%, 98%, 99% or higher relative to the disclosed sequences.

“Identity” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent identity, and not considering any conservative substitutions as part of the sequence identity. Neither N- or C-terminal extensions nor insertions shall be construed as reducing identity. Methods and computer programs for the alignment are well known. The percent identity can be determined by standard alignment algorithms, for example, the Basic Local Alignment Search Tool (BLAST) described by Altshul et al. ((1990) J. Mol. Biol., 215: 403 410); the algorithm of Needleman et al. ((1970) J. Mol. Biol., 48: 444 453); or the algorithm of Meyers et al. ((1988) Comput. Appl. Biosci., 4: 11 17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

“Amino acid(s)” refer to all naturally occurring L-α-amino acids, e.g., and include D-amino acids. The phrase “amino acid sequence variant” refers to molecules with some differences in their amino acid sequences as compared to the sequences according to the present disclosure Amino acid sequence variants of an antibody according to the present disclosure, e.g., of a specified sequence, still have the ability to bind the IgE. Amino acid sequence variants include substitutional variants (those that have at least one amino acid residue removed and a different amino acid inserted in its place at the same position in a polypeptide according to the present disclosure), insertional variants (those with one or more amino acids inserted immediately adjacent to an amino acid at a particular position in a polypeptide according to the present disclosure) and deletional variants (those with one or more amino acids removed in a polypeptide according to the present disclosure).

The term “pharmaceutically acceptable” means a nontoxic material that does not interfere with the effectiveness of the biological activity of the active ingredient(s).

The term “administering” in relation to a compound, e.g., an anti-IgE antibody, is used to refer to delivery of that compound to a patient by any route.

As used herein, a “therapeutically effective amount” refers to an amount of anti-IgE antibody (e.g., omalizumab or an antigen-binding fragment thereof) that is effective, upon single or multiple dose administration to a patient (such as a human) for treating, preventing, preventing the onset of, curing (if applicable), delaying, reducing the severity of, ameliorating at least one symptom of a disorder or recurring disorder, or prolonging the survival of the patient beyond that expected in the absence of such treatment. When applied to an individual active ingredient (e.g., an anti-IgE antibody, e.g., omalizumab) administered alone, the term refers to that ingredient alone. When applied to a combination, the term refers to combined amounts of the active ingredients that result in the therapeutic effect, whether administered in combination, serially or simultaneously.

The term “treatment” or “treat” is herein defined as the application or administration of an anti-IgE antibody according to the disclosure, for example, omalizumab, or a pharmaceutical composition comprising said anti-IgE antibody, to a subject or to an isolated tissue or cell line from a subject, where the subject has a particular disease, a symptom associated with the disease, or a predisposition towards development of the disease, where the purpose is to cure (if applicable), delay the onset of, reduce the severity of, alleviate, ameliorate one or more symptoms of the disease, improve the disease, reduce or improve any associated symptoms of the disease or the predisposition toward the development of the disease. The term “treatment” or “treat” includes treating a patient suspected to have the disease as well as patients who are ill or who have been diagnosed as suffering from the disease or medical condition, and includes suppression of clinical relapse.

As herein defined, the term “diseases or disorders involving dyfunctioning of regulatory T cells” refers e.g. to diseases involving disorders of regulatory T cells immunomodulation.

Anti-IgE Antibodies

In some embodiments of the disclosed uses, methods, and kits, the anti-IgE antibody or antigen-binding fragment thereof is a monoclonal antibody. In some embodiments, the anti-IgE antibody or antigen-binding fragment thereof is a human or humanized antibody. In some embodiments, the anti-IgE antibody or antigen-binding fragment thereof is a humanized antibody. In some embodiments, the anti-IgE antibody or antigen-binding fragment thereof is a human antibody of the IgG₁ subtype. In some embodiments, the anti-IgE antibody or antigen-binding fragment thereof is omalizumab. In other embodiments, the anti-IgE antibody or antigen-binding fragment thereof is ligelizumab.

Exemplary anti-IgE antibodies include, but are not limited to, omalizumab, quilizumab, ligelizumab and etrolizumab.

Alternatively, an anti-IgE antibody or antigen-binding fragment thereof used in the disclosed methods may be an amino acid sequence variant of the reference anti-IgE antibodies set forth herein.

The disclosure also includes anti-IgE antibodies or antigen-binding fragments thereof (e.g., omalizumab) in which one or more of the amino acid residues of the V_{H} or V_{L} domain of omalizumab, typically only a few (e.g. 1-10), are changed; for instance by mutation, e.g., site directed mutagenesis of the corresponding DNA sequences.

Methods of Treatment and Uses of Anti-IgE Antibodies

The disclosed anti-IgE antibody (e.g., omalizumab) or antigen-binding fragment thereof, may be used in vitro, ex vivo, or incorporated into pharmaceutical compositions and administered in vivo to treat patients (e.g., human patients) affected by one or more disease or disorder involving Treg cells dysfunction.

The anti-IgE antibody (e.g, omalizumab) or antigen-binding fragment thereof may be used as a pharmaceutical composition when combined with a pharmaceutically acceptable carrier. Such a composition may contain, in addition to the anti-IgE antibody or antigen-binding fragment thereof, carriers, various diluents, fillers, salts, buffers, stabilizers, solubilizers, and other materials well known in the art. The characteristics of the carrier will depend on the route of administration.

Pharmaceutical compositions for use in the disclosed methods may be manufactured in conventional manner. In one embodiment, the pharmaceutical composition is provided in lyophilized form. For immediate administration it is dissolved in a suitable aqueous carrier, for example sterile water for injection or sterile buffered physiological saline. Other formulations comprise liquid or lyophilized formulation.

Antibodies, e.g., antibodies to IgE, or antigen-binding fragment thereof, are typically formulated either in aqueous form ready for parenteral administration or as lyophilisates for reconstitution with a suitable diluent prior to administration. In some embodiments of the disclosed methods and uses, the anti-IgE antibody or antigen-binding fragment thereof, e.g., omalizumab, is formulated as a lyophilisate. Suitable lyophilisate formulations can be reconstituted in a small liquid volume (e.g., 2 ml or less, e.g., 1 ml) to allow subcutaneous administration and can provide solutions with low levels of antibody aggregation. Techniques for purification of antibodies to a pharmaceutical grade are well known in the art.

Disclosed methods of, and anti-IgE antibodies for use in, preventing, treating or modifying the course of a disease or disorder involving dysfunctioning of Treg cells in a patient in need thereof, comprising herein are administering the patient a therapeutically effective amount of ananti-IgE antibody or antigen-binding fragment thereof.

For example, diseases or disorders involving dysfunctioning of Treg cells are selected from the group consisting of type 1 diabetes, glomerulonephritis, allergic encephalomyelitis, multiple sclerosis, systemic lupus erythematosus, inflammatory bowel diseases, autoimmune gastritis, myasthenia gravis, autoimmune thyroiditis, acquired aplastic anemia auto-immune encephalitis, Parkinson's disease, Foxp3− deficiency, IPEX syndrome, immuno-dysregulation, polyendocrinopathy, enteropathy, anti-tumor immunity, and transplant rejection.

In another example, the disease or disorder involving dysfunctioning of Treg cells refers to rheumatic arthritis.

In yet another example, the disease or disorder involving dysfunctioning of Treg cells refers to diseases or disorders selected from type 1 diabetes, glomerulonephritis, allergic encephalomyelitis, inflammatory bowel diseases, autoimmune gastritis, myasthenia gravis, acquired aplastic anemia, auto-immune encephalitis, Parkinson's disease, Foxp3-deficiency, IPEX syndrome, immuno-dysregulation, enteropathy, anti-tumor immunity, and transplant rejection.

In yet another example, the disease or disorder involving dysfunctioning of Treg cells refers to multiple sclerosis, autoimmune thyroiditis or polyendocrinopathy.

Disclosed herein are methods of, and anti-IgE antibodies (e.g omalizumab) for use in, preventing, treating or modifying the course of a disease or disorder involving dysfunctioning of Treg cells in a patient in need thereof, comprising administering the patient a therapeutically effective amount of anti-IgE antibody or antigen-binding fragment thereof.

The patient in need thereof may have an insulin allergy.

In another embodiment, the patient is affected by asthma, e.g. allergic asthma.

In another example, the patient is affected by urticaria, e.g. chronic spontaneous urticaria (CSU).

In another embodiment, the patient is affected by rhinitis, e.g. allergic rhinitis.

In yet another embodiment, the patient is affected by a disease or disorder selected from allergy, asthma, urticarial and rhinitis, e.g. a disease or condition selected from allergic asthma, CSU and allergic rhinitis.

Furthermore, disclosed herein are methods of, and anti-IgE antibodies or antigen-binding fragment thereof, e.g omalizumab, for use in, preventing, treating or modifying the course of a disease or disorder involving dysfunctioning of Treg cells in a patient in need thereof, comprising administering the patient a therapeutically effective amount of anti-IgE antibody or antigen-binding fragment thereof, wherein the patient is not affected by allergy, asthma, urticarial or rhinitis. For example, the patient may not be allergic, or may not have an allergic condition selected from allergic asthma, CSU, allergic rhinitis.

In one embodiment, the patient in need thereof has no allergy.

In another embodiment, the patient is not affected by asthma, e.g. allergic asthma.

In another example, the patient is not affected by urticaria, e.g. chronic spontaneous urticaria (CSU).

In another embodiment, the patient is not affected by rhinitis, e.g. allergic rhinitis.

The appropriate dosage will vary depending upon, for example, the particular anti-IgE antibody to be employed, the host, the mode of administration and the nature and severity of the condition being treated, and on the nature of prior treatments that the patient has undergone. It may also depend on the level of IgE in the patient's blood before initiating the treatment with the anti-IgE antibody.

Ultimately, the attending health care provider will decide the amount of the anti-IgE antibody with which to treat each individual patient. In some embodiments, the attending health care provider may administer low doses of the anti-IgE antibody and observe the patient's response, in particle the blood level of IgE.

For the asthma indication, the usual dose range of omalizumab is between 75 mg and 600 mg in one to four subcutaneously injections, and the maximum recommended dose is 600 mg. The dosing of omalizumab for treating asthma is determined based on the patient's weight and the patient's serum total IgE level. The dosage of omalizumab for chronic urticaria indication is 300 mg sc per month.

In one embodiment of the present disclosure, omalizumab is adminstered subcutaneously at a dose of about 75 mg to about 600 mg, e.g at a dose of about 300 mg, e.g at a maximum dose of 600 mg.

In some embodiments, the level of IgE in the patient's blood is measured before initiating the administration of the anti-IgE antibody, e.g., omalizumab, and the dose of the antibody is adjusted based on the weight of the patient and/or his serum total IgE level.

The duration of therapy using a pharmaceutical composition of the present disclosure will vary, depending on the severity of the disease or disorder to be treated and the condition and personal response of each individual patient. In some embodiments, the patient is administered the anti-IgE antibody (e.g., omalizumab) for long-term, e.g. at least 12 weeks, e.g. up to 16 weeks, e.g. to 12 to 16 weeks.

In some embodiments, the anti-IgE antagonist (e.g., omalizumab) is administered to the patient every two weeks, e.g. every two or four weeks, e.g monthly.

The anti-IgE antibody or antigen-binding fragment thereof according to the present disclosure, e.g., omalizumab, is conveniently administered parenterally, e.g., intravenously, intramuscularly, or subcutaneously, e.g. subcutaneously.

The anti-IgE antibody or antigen-binding fragment thereof, e.g., omalizumab, may be administered to the patient subcutaneously (SC), e.g. at about 75 mg to about 600 mg (e.g. about 75 mg, about 600 mg), e.g. at about 300 mg.

In practicing some of the methods of treatment or uses of the present disclosure, a therapeutically effective amount of an anti-IgE antibody (e.g., omalizumab) or antigen-binding fragment thereof, is administered to a patient, e.g., a mammal (e.g., a human). While it is understood that the disclosed methods provide for treatment of diseases or disorders involving involving Treg cells dysfunctioning, using anti-IgE antibody (e.g., omalizumab) or antigen-binding fragment thereof this does not preclude that, if the patient is to be ultimately treated with an anti-IgE antibody (e.g., omalizumab) or antigen-binding fragment thereof, therapy is necessarily a monotherapy.

Indeed, if a patient is selected for treatment with an anti-IgE antibody or antigen-binding fragment thereof, then the anti-IgE antibody (e.g., omalizumab) or antigen-binding fragment thereof, may be administered in accordance with the methods of the disclosure either alone or in combination with other agents and therapies for treating the patient affected by the disease or disorder involving Treg cells dysfunctioning, e.g., in combination with at least one additional therapeutic agent, such as e.g., a corticosteroid or an immumosuppressor, e.g., a systemic corticosteroid or an immunosuppressor.

That can be the case for example, when the patient to be treated is allergic, or when the patient is also affected by another disease or disorder selected from asthma, urticaria, and rhinitis, e.g. selected from allergic asthma, CSU, and allergic rhinitis.

When coadministered with one or more additional psoriasis agent(s), the anti-IgE antibody or antigen-binding fragment thereof may be administered either simultaneously with the other agent, or sequentially. If administered sequentially, the attending physician will decide on the appropriate sequence of administering the anti-IgE antibody or antigen-binding fragment thereof in combination with other agents and the appropriate dosages for co-delivery.

Various therapies may be beneficially combined with the disclosed anti-IgE antibodies, such as omalizumab, during treatment of the disease or disorder involving Treg cells dysfunctioning disclosed herein. Such therapies include for example corticosteroids (e.g., systemic corticosteroids) or immunosuppressors.

Disclosed herein are methods of, and anti-IgE antibodies (e.g, omalizumab) or antigen-binding fragment thereof, for use in, modifying the course of a disease or disorder involving Treg cells dysfunctioning, in a patient in need thereof, comprising administering the patient a dose of about 75 mg to about 600 mg of an anti-IgE antibody (e.g., omalizumab) or antigen-binding fragment thereof by subcutaneous injection.

In some embodiments of the disclosed uses, methods, and kits, the patient has allergy, asthma and/or urticarial, e.g, has a disease or disorder selected from asthma, allergic asthma, rhinitis, allergic rhinitis, urticarial and CSU. In some embodiments of the disclosed uses, methods, and kits, the patient has allergic asthma, allergic urticaria and/or CSU. In other embodiments of the disclosed uses, methods, and kits, the patient has allergic asthma and CSU.

In some embodiments of the disclosed uses, methods, and kits, the anti-IgE antibody (e.g., omalizumab) or antigen-binding fragment thereof, can be prescribed as first treatment or added on to any of the standard of care medications

Kits

The disclosure also encompasses kits for treating particular patients having disease or disorder involving Treg cells dysfunctioning. Such kits comprise an anti-IgE antibody (e.g., omalizumab) or antigen-binding fragment thereof, (e.g., in liquid or lyophilized form) or a pharmaceutical composition comprising the anti-IgE antibody (described supra). Additionally, such kits may comprise means for administering the anti-IgE antibody or antigen-binding fragment thereof (e.g., an auto-injector, a syringe and vial, a prefilled syringe, a prefilled pen) and instructions for use. These kits may contain additional therapeutic agents (described supra) for treating the disease or disorder involving Treg cells dysfunctioning, e.g., for delivery in combination with the enclosed anti-IgE antibody or antigen-binding fragment thereof, e.g., omalizumab. Such kits may also comprise instructions for administration of the anti-IgE antibody or antigen-binding fragment thereof, (e.g., omalizumab.) to treat the patient. Such instructions may provide the dose (e.g., 75 mg, 300 mg), route of administration (e.g., IV, SC), and dosing regimen (e.g., every tow or four weeks during e.g. 12 to 16 weeks) for use with the enclosed anti-IgE antibody or antigen-binding fragment thereof, e.g., omalizumab.

The phrase “means for administering” is used to indicate any available implement for systemically administering a drug to a patient, including, but not limited to, a pre-filled syringe, a vial and syringe, an injection pen, an auto-injector, an IV drip and bag, a pump, etc. With such items, a patient may self-administer the drug (i.e., administer the drug without the assistance of a physician) or a medical practitioner may administer the drug.

The term “non-responder” to therapy using corticosteroids or other conventional therapy, is defined as a subject who failed to achieve at least 50%, e.g. 50%-90% improvement of their baseline or had an exacerbation of their symptoms. Responder to therapy using corticosteroids or other conventional therapy, is defined as a subject who achieved least 50%, e.g. 50%-90%, e.g. 90% improvement of baseline.

Disclosed herein are kits for use in modifying the disease course in a patient having disease or disorder involving Treg cells dysfunctioning, comprising an anti-IgE antibody (e.g., omalizumab) or antigen-binding fragment thereof. In some embodiments, the kit further comprises means for administering the anti-IgE antibody (e.g, omalizumab) or antigen-binding fragment thereof, to the patient.

### EMBODIMENTS

1. An anti-IgE antibody or antigen binding fragment thereof for use in treating or preventing a disease or disorder involving Treg cells dysfunctioning in a subject in need thereof.
2. A method of treating a disease or disorder involving Treg cells dysfunctioning, comprising administering a subject in need thereof a therapeutically effective amount of an anti-IgE antibody or antigen binding fragment thereof.
3. The anti-IgE antibody or antigen binding fragment thereof according to embodiment 1 or the method according to embodiment 2, wherein the subject in need thereof is a not-responder to treatment with corticosteroids.
4. The anti-IgE antibody or antigen binding fragment thereof according to embodiment 1 or the method according to embodiment 2, wherein the subject in need thereof is a subject with an inadequate response to corticosteroids, or is in need of corticosteroid-sparing, or in whom corticosteroid treatment is inappropriate.
5. The anti-IgE antibody or antigen binding fragment thereof according to embodiment 1 or the method according to embodiment 2, wherein the subject in need thereof has had an inadequate response to conventional therapy including corticosteroids, or is intolerant to or has medical contraindications for such therapies.
6. The anti-IgE antibody or antigen binding fragment thereof according to embodiment 1 or the method according to embodiment 2, wherein the subject in need thereof cannot tolerate a treatment with corticosteroids.
7. The anti-IgE antibody or antigen binding fragment thereof according to embodiment 1 or the method according to embodiment 2, wherein the subject in need thereof has an inadequate response to corticosteroids.
8. The anti-IgE antibody or antigen binding fragment thereof according to any one of the preceding embodiments, wherein the disease or disorder is selected from type 1 diabetes, glomerulonephritis, allergic encephalomyelitis, multiple sclerosis, inflammatory bowel diseases, autoimmune gastritis, myasthenia gravis, autoimmune thyroiditis, acquired aplastic anemia, auto-immune encephalitis, Parkinson's disease, Foxp3− deficiency, IPEX syndrome, immuno-dysregulation, polyendocrinopathy, enteropathy, anti-tumor immunity, or transplant rejection.
9. The anti-IgE antibody or antigen binding fragment thereof or the method according to any one of the preceding embodiments, wherein the disease or disorder is selected from type 1 diabetes, glomerulonephritis, allergic encephalomyelitis, inflammatory bowel diseases, autoimmune gastritis, myasthenia gravis, acquired aplastic anemia, auto-immune encephalitis, Parkinson's disease, Foxp3-deficiency, IPEX syndrome, immuno-dysregulation, enteropathy, anti-tumor immunity, or transplant rejection.
10. The anti-IgE antibody or antigen binding fragment thereof or method according to any of the above embodiments, wherein the patient is further affected by a disease or condition selected from allergy, asthma, urticarial and rhinitis, e.g. a disease or condition selected from allergic asthma, chronic spontaneous urticaria and allergic rhinitis.
11. The anti-IgE antibody or antigen binding fragment thereof or method according to according to any one of the preceding embodiments, wherein the patient is not affected by allergy, asthma, urticarial or rhinitis, e.g. is not affected by allergic asthma, chronic spontaneous urticaria or allergic rhinitis.
12. The anti-IgE antibody or antigen binding fragment thereof or method according to any one of the preceding embodiments, wherein the anti-IgE antibody is omalizumab or ligelizumab.
13. The anti-IgE antibody or antigen binding fragment thereof or method according to embodiment 12, wherein omalizumab is administered at a dose of about 75 mg to about 600 mg, e.g. at a maximum dose of 600 mg.
14. The anti-IgE antibody or antigen binding fragment thereof or method according to embodiment 12 or embodiment 13, wherein omalizumab is administered every two to four weeks.
15. The anti-IgE antibody or antigen binding fragment thereof or method according to any one of embodiments 12 to 14, wherein omalizumab is administered during up to 16 weeks, e.g. 12 to 16 weeks.
16. The anti-IgE antibody or antigen binding fragment thereof or method according to any of the above embodiments, wherein the anti-IgE antibody is co-administered with a corticosteroid and/or an immunusuppressor.

General

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure. These examples should in no way be construed as limiting the scope of the disclosed patient matter, as defined by the appended claims.

### EXAMPLES

### Example 1. Plasmacytoid Dendritic Cells as a Suitable In Vitro Model

1.1. Plasmacytoid Dendritic Cells Purification and Characterization.

Plasmacytoid dendritic cells (pDCs) are capable of inducing functional regulatory T cells (Treg) upon TLR9-ligand (TLR9-L) stimulation, a common stimulus encountered after bacterial infections.

Human pDCs were purified to homogeneicity from peripheral blood mononuclear cells (PBMCs) from buffy coats by using the Plasmacytoid Dendritic Cell Isolation Kit II (Miltenyi Biotec).

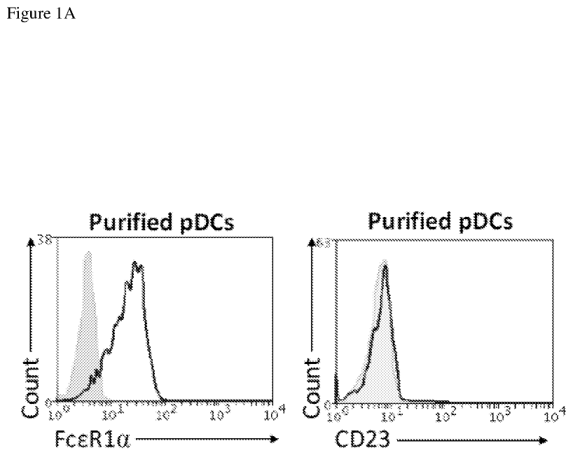

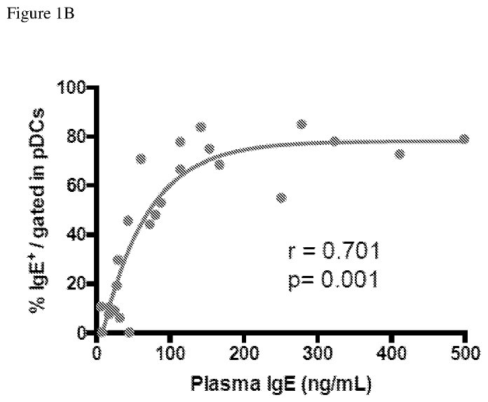

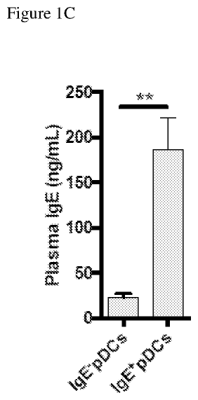

The purity of isolated pDCs ranged between 85-95%. As shown in FIG. 1A, purified human pDCs expressed high levels of the high affinity IgE receptor (FcεR1) with minimal expression of the low affinity IgE receptor (CD23).

The levels of plasma IgE from the different donors was quantified by ELISA. There was a positive correlation between plasmatic IgE levels and the frequency of pDCs displaying IgE bound to FcεR1 (FIG. 1B).

The mean value of the percentage of pDCs displaying IgE bound to FcεR1 when considering all the assayed donors was 45.71±6.23 (mean±SEM). Considering this value as a cut-off to differentiate non-atopic versus atopic donors, for further experiments those donors whose pDCs displayed FcεR1-bound IgE frequencies higher than 45% (IgE+pDCs or atopic donors) were selected.

The levels of plasma IgE in the atopic donors included in the study for further detailed analysis in this study (IgE+pDCs frequencies higher than 45%) were significantly higher than those from non-atopic donors (IgE− pDCs) (p=0.002, FIG. 1C).

### Example 2. IgE-Mediated FcεR1-Crosslinking Reduces the Transcription/Expression of Functional Markers and Cytokines Essential for Treg Cells Induction

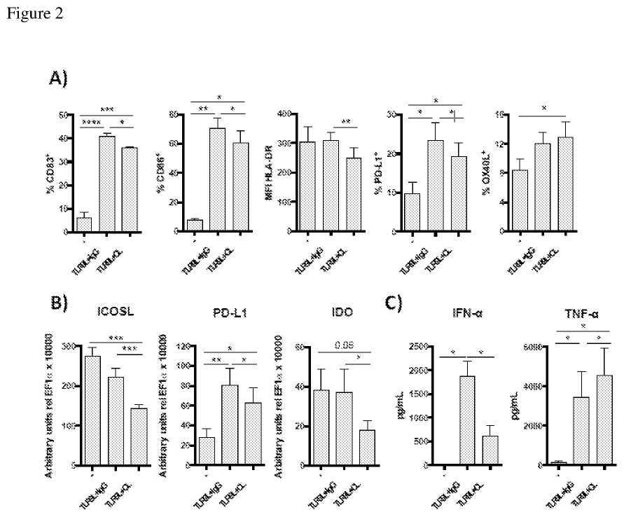

IgE-mediated FcεR1-crosslinking initiates downstream signalling in dendritic cells (DCs) with functional implications. To induce the IgE-FcεR1 complex activation, a rabbit anti-human IgE (IgE-FcεR1-crosslinker, CL) was used. Purified pDCs were stimulated with 10 μg/mL of IgE-FcεR1-crosslinker (CL) or the corresponding isotype control (IgG) for 1 hour followed by the addition of 2 μM of CpG type B, ODN2006 (TLR9-L). After 18 hours of stimulation, pDCs gene expression, activation/functional markers expression and supernatant cytokines were analysed (FIG. 2). IgE-mediated FcεR1-crosslinking significantly reduced the frequency of CD83 (p=0.033), CD86 (p=0.046), PD-L1 (p=0.05) and the expression of HLA-DR (p=0.006) in TLR9-L-activated pDCs compared to the isotype control (FIG. 2A). Additionally, IgE-mediated FcεR1-crosslinking also decreased the expression of ICOSL (p=0.003), PD-L1 (p=0.048), IDO genes (p=0.05) (FIG. 2B) and the secretion of IFN-□ (p=0.022) (FIG. 2C). All these molecules are known to play a critical role in the generation of Treg cells. Similarly, IgE-mediated FcεR1-crosslinking also significantly decreased the expression of SOCS1 and IKβα (data not shown). In contrast, IgE-mediated FcεR1-crosslinking signalling increased OX40L cell frequency (p=0.025) (FIG. 2A) in TLR9-L-activated pDCs compared to the unstimulated condition and TNF-α secretion compared to either the unstimulated (p=0.017) and stimulated condition (p=0.041, FIG. 2C).

### Example 3. Capacity of Human TLR9-L-Activated pDCs to Generate Treg Cells after IgE-Mediated FcεR1-Crosslinking

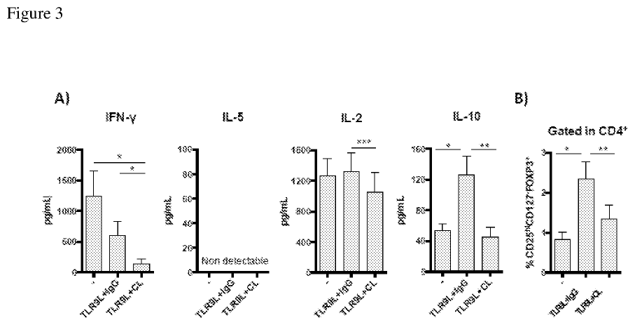

Purified pDCs were stimulated with TLR9-L and cocultured with allogeneic naïve CD4+ T-cells in a 1:5 ratio (pDCs: naïve T-cel) in the absence or presence of IgE-FcεR1-crosslinker. TLR9-L-activated pDCs induced Treg cells polarization, as identified from the increase in IL-10 secretion (p=0.041) (FIG. 3A), and also from the increase in the frequency of CD4+CD127lowCD25+Foxp3+ Treg cells (p=0.018) (FIG. 3B). IgE-mediated FcεR1-crosslinking impaired the capacity of TLR9-L-activated pDCs to induce Treg cells polarization (p=0.009, FIG. 3B), which was accompanied by the significant decrease of IL-10 in the coculture supernatant (p=0.002, FIG. 3A). IgE-mediated FcεR1-crosslinking also induced a decrease in IFN-α (p=0.029) and IL-2 (p>0.001, FIG. 3A).

### Example 4. Omalizumab Removes Membrane-Bound IgE on Purified pDCs from Atopic Donors

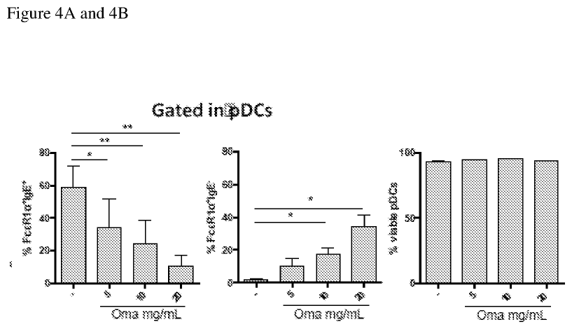

Omalizumab reduced the IgE bound to FcεR1 on pDCs, as shown by the decrease in IgE mean fluorescence intensity (MFI) in a dose-dependent manner on purified pDCs. Consequently, there was a significant reduction in the frequency of pDCs with FcεR1-bound IgE (FcεR1+IgE+pDCs) and concomitant increased levels of pDCs with unoccupied FcεR1 (FcεR1+IgE-pDCs) (FIG. 4A). Omalizumab did not affect pDCs viability (FIG. 4B). Summing up, omalizumab removes IgE from FcεR1 in pDCs in vitro.

### Example 5

Omalizumab restores the cytokine signature in TLR9-L-activated pDCs in the presence of IgE-mediated FcεR1-crosslinking. pDCs were treated with 5 and 10 mg/mL of omalizumab for 24 hours prior to IgE-FcεR1-crosslinker and TLR9-L stimulation. Then, the cytokine profile was analysed by ELISA 24 hours after activation. Omalizumab at 10 mg/mL restored pDCs capacity to produce IFN-α compared to the IgE-FcεR1-crosslinker condition (p=0.05) (FIG. 5A). In line with such result, pDCs treated with 10 mg/mL retained low TNF-α levels compared to the IgE-FcεR1-crosslinker condition (p=0.04) (FIG. 5B). Summing up, IgE-FcεR1-crosslinker inhibited IFN-α and induced TNF-α production in TLR9-L-activated pDC, which is restored to normal levels when pDC were previously treated with omalizumab.

### Example 6. Capacity of Omalizumab to Promote the Generation of Treg Cells in Comparison to Corticosteroids

To check whether omalizumab could restore the capacity of pDCs to generate Treg cells in comparison with the effects exerted by corticosteroids, pDCs were treated with 5 or 10 mg/mL of omalizumab for 24 hours. pDCs were then treated with 1 μM Dexamethasone, IgE-FcεR1-crosslinker and TLR9-L stimulation. After 24 hours, the pDCs were washed and cocultured with naïve CD4+ T cells for 5 days. There was a significant increase of Treg cells associated to the TLR9-L stimulation (p=0.004) (FIG. 6), which was abolished by Dexamethasone treatment. IgE-FcεR1-crosslinker decreased the frequency of induced Treg cells (p=0.049), which was reverted by Omalizumab in a dose dependent manner. Summing up, omalizumab is capable of restoring pDCs capacity to generate Treg cells under IgE-FcεR1-crosslinker stimulation, whereas Dexamethasone suppress pDCs function.

### Example 7. IgE-Mediated FcεR1-Crosslinking Impairs IDO, PD-L1 and IFN-α, which Contribute to the Capacity of pDCs to Generate Treg Cells

IgE-mediated FcεR1-crosslinking reduces the expression of PD-L1, IDO and the secretion of IFN-α by TLR9-L activated pDCs. Since the capacity to induce Treg cells by TLR9-L activated pDCs was impaired under IgE-FcεR1-crosslinker conditions, blocking experiments were performed to elucidate whether the downregulation of IDO, PD-L1 and IFN-α might be associated to pDCs impairment in Treg cells induction. Indoleamine-2,3 dioxygenase (IDO) generates kynurenine (kyn) from tryptophan, which is involved in the generation of Treg cells. pDCs capacity to induce Treg cells under IgE-FcεR1-crosslinker condition increased when kyn was added to the coculture (p=0.07) (FIG. 7), which demonstrates that kyn bypassed IDO downregulation and highlights the relevance of IDO expression in pDCs and its role in Treg cells generation. To further demonstrate the role of IDO in Treg generation, 1-methyl-D-tryptophan (1-MT) which has inhibitory effects over IDO enzymatic activity, was added to the coculture. 1-MT impaired TLR9-L-activated pDC capacity to generate Treg cells (p=0.015), which strengthens that IDO downregulation, associated to IgE-mediated FcεR1-crosslinking, is partially responsible for the reduced Treg cells-generation under IgE-FcεR1-crosslinker conditions.

Programmed cell death ligand-1 (PD-L1) expressed in pDC plays a critical role in the generation of Treg cells through the PD-1/PD-L1 axis interaction. Blocking PD-L1 through anti-human PD-L1 partially inhibited TLR9-L activated pDCs capacity to induce Treg cells. Therefore, PD-L1 downregulation associated to IgE-mediated FcεR1-crosslinking, is also responsible for the decrease in Treg cells generation. The combination of 1-MT and anti-PD-L1 had additive effects, and abrogated the Treg cells generation to FcεR1-crosslinking condition levels.

Some authors suggest that IDO upregulation in pDCs is partially dependent on the autocrine effect of IFN-α secretion upon TLR9-L activation. Additionally, it is claimed that IFN-α itself induces the generation of Treg cells. Since the secretion of IFN-α by TLR9-L activated pDCs was impaired upon IgE-mediated FcεR1-crosslinking, blocking experiments using anti-IFNAR were performed to check whether pDCs IFN-α secretion may be responsible for the decrease in Treg cells generation observed after IgE-mediated FcεR1-crosslinking. There was a decrease in Treg cells generation when IFN-α receptor was blocked (FIG. 7). Therefore, the decrease in IFN-α secretion associated to IgE-mediated FcεR1-crosslinking in TLR9-L activated pDCs, might also contribute to the impairment of Treg cell induction by pDCs.

The dendritic cells (DCs) induce the regulatory T cells (Treg), which when induced, are essential for healthy immune responses to allergens and to prevent airway remodelling. IgE blocks the FcεR1 on the DCs and hence reduces this TReg induction.

Omalizumab may restore the capacity of the dendrtic cells to induce the regulatory T cells caused by the interaction between IgE and the FcεR1.

This is been proven in vitro where dendritic cells were purified and incubated with omalizumab, then activated by CpG ODN (TLR9-L) in a presence of IgE-FcεR1 crosslinker, then the effects on the TReg cells was assessed by ELISA, PCR and cultures.

While corticosteroids abolish the capacity of DCs to induce TReg, omalizumab was able to induce them, proving one more time that omalizumab can have well tolerated immuno-modulatory effect compared to a known low safety profile immunosuprression by corticosteroids.

What is claimed is:

1. An anti-IgE antibody or antigen binding fragment thereof for use in treating or preventing a disease or disorder involving Treg cells dysfunctioning in a subject in need thereof.

2. A method of treating a disease or disorder involving Treg cells dysfunctioning, comprising administering a subject in need thereof a therapeutically effective amount of an anti-IgE antibody or antigen binding fragment thereof.

3. The anti-IgE antibody or antigen binding fragment thereof according to claim 1 or the method according to claim 2, wherein the subject in need thereof is a not-responder to treatment with corticosteroids.

4. The anti-IgE antibody or antigen binding fragment thereof according to claim 1 or the method according to claim 2, wherein the subject in need thereof is a subject with an inadequate response to corticosteroids, or is in need of corticosteroid-sparing, or in whom corticosteroid treatment is inappropriate.

5. The anti-IgE antibody or antigen binding fragment thereof according to claim 1 or the method according to claim 2, wherein the subject in need thereof has had an inadequate response to conventional therapy including corticosteroids, or is intolerant to or has medical contraindications for such therapies.

6. The anti-IgE antibody or antigen binding fragment thereof according to claim 1 or the method according to claim 2, wherein the subject in need thereof cannot tolerate a treatment with corticosteroids.

7. The anti-IgE antibody or antigen binding fragment thereof according to claim 1 or the method according to claim 2, wherein the subject in need thereof has an inadequate response to corticosteroids.

8. The anti-IgE antibody or antigen binding fragment thereof according to any one of the preceding claims, wherein the disease or disorder is selected from type 1 diabetes, glomerulonephritis, allergic encephalomyelitis, multiple sclerosis, inflammatory bowel diseases, autoimmune gastritis, myasthenia gravis, autoimmune thyroiditis, acquired aplastic anemia, auto-immune encephalitis, Parkinson's disease, Foxp3− deficiency, IPEX syndrome, immuno-dysregulation, polyendocrinopathy, enteropathy, anti-tumor immunity, or transplant rejection.

9. The anti-IgE antibody or antigen binding fragment thereof or the method according to any one of the preceding claims, wherein the disease or disorder is selected from type 1 diabetes, glomerulonephritis, allergic encephalomyelitis, inflammatory bowel diseases, autoimmune gastritis, myasthenia gravis, acquired aplastic anemia, auto-immune encephalitis, Parkinson's disease, Foxp3-deficiency, IPEX syndrome, immuno-dysregulation, enteropathy, anti-tumor immunity, or transplant rejection.

10. The anti-IgE antibody or antigen binding fragment thereof or method according to any of the above claims, wherein the patient is further affected by a disease or condition selected from allergy, asthma, urticarial and rhinitis, e.g. a disease or condition selected from allergic asthma, chronic spontaneous urticaria and allergic rhinitis.

11. The anti-IgE antibody or antigen binding fragment thereof or method according to any one of the preceding claims, wherein the patient is not affected by allergy, asthma, urticarial or rhinitis, e.g. is not affected by allergic asthma, chronic spontaneous urticaria or allergic rhinitis.

12. The anti-IgE antibody or antigen binding fragment thereof or method according to any one of the preceding claims, wherein the anti-IgE antibody is omalizumab or ligelizumab.

13. The anti-IgE antibody or antigen binding fragment thereof or method according to claim 12, wherein omalizumab is administered at a dose of about 75 mg to about 600 mg, e.g at a maximum dose of 600 mg.

14. The anti-IgE antibody or antigen binding fragment thereof or method according to claim 12 or claim 13, wherein omalizumab is administered every two to four weeks.

15. The anti-IgE antibody or antigen binding fragment thereof or method according to any one of claims 12 to 14, wherein omalizumab is administered during up to 16 weeks, e.g. 12 to 16 weeks.

16. The anti-IgE antibody or antigen binding fragment thereof, or method according to any of the above claims, wherein the anti-IgE antibody is co-administered with a corticosteroid and/or an immunusuppressor.
